(19) United States
(12) Patent Application Publication
(10) Pub. No.: US 2021/0045890 A1
(43) Pub. Date: Feb. 18, 2021

(54) COMBINED SEMI-LIMITING MULTIPOLAR ARTIFICIAL WRIST JOINT

(71) Applicant: Hongwen ZHU, Tianjin (CN)

(72) Inventors: HONGWEN ZHU, Tianjin (CN); GUOFU HUANG, Tianjin (CN); RONGHUA DONG, Tianjin (CN); TIANMOU ZHU, Tianjin (CN)

(21) Appl. No.: 17/088,451

(22) Filed: Nov. 3, 2020

(30) Foreign Application Priority Data
Nov. 6, 2019 (CN) 201911078221X

(51) Int. Cl. A61F 2/42

(52) U.S. Cl. CPC A61F 2/4261; A61F 2002/3065

(57) ABSTRACT

The present invention discloses a combined semi-limiting multipolar artificial wrist joint, or “semi-limiting bipolar artificial wrist joint”, and be selected and assembled based on the specific pathological conditions of wrist bone destruction. For instance, the metacarpal bone articular surface is shown to be complete and the bone quality is good, and most of the far carpal bones (including small polyangular bones and head bones) can be preserved, while the proximal carpal bones (scaphoid and lunate) and the articular surface of the carpal radius are broken, and the proximal carpal bones are to be excised. The artificial wrist joint can also be assembled in another way, which is called “semi-limiting multipolar artificial wrist joint”, wherein the pathologically damaged double carpal bones are excised to form an inter-matching assembly of palm, wrist and radius joints.

### TECHNICAL FIELD

The invention relates to a medical artificial joint prosthesis, particularly relates to a combined semi-limiting multipolar artificial wrist joint in the field of hand surgery.

### BACKGROUND ART

At present, the use of artificial joint replacement surgery to treat and repair the damage of human joints is one of the effective methods that the international medical community agrees. Especially in recent years, due to the continuous development of medical technology, the emergence of new biomaterials, and the rapid development of high-tech, the replacement and repair of human joint damage and dysfunction have achieved significant and good clinical results. The application of human hip and knee replacement surgery has been widely promoted and used by doctors in my country, and the satisfactory clinical effect has been accepted by the majority of patients.

In response to the demand for human micro joint prosthesis, clinical development is urgently needed, especially in the field of hand surgery. Many diseases cause bone destruction, deformity and dysfunction of the wrist, which seriously affect people's quality of life. At present, orthopedics and functional reconstruction technology by artificial joints is expected to develop rapidly in the medical field of China.

The existing micro-joint prostheses mostly rely on imported products which have few types and varieties, and not enough models can be matched. It has not yet been accepted and used by the majority of medical units in many regions.

The incidence of rheumatism and rheumatoid arthritis worthy of attention is still relatively high in China. The destruction, deformity and dysfunction of the small joints of the limbs can be said to be the “patent” of rheumatoid, which are difficult to be cured by medications and bring the patient great pain.

The human wrist joints are complex, and have various bone shapes and a large number of bones. The joint surfaces have multi-directional anastomosis and functional movement can be universal. Once the damage, deformity and dysfunction are caused by trauma and related diseases, an ideal replacement treatment of artificial joints is a solution worth promoting in medical treatment.

The wrist joint has a unique and complex anatomical relationship. It is a multi-directional freedom joint composed of the distal end face of the ulna radius, the proximal and distal carpal bones and the proximal end face of the metacarpal bone. It has biomechanical functions of strong flexion and extension, lateral deviation, and internal and external rotation.

According to the specific pathological changes of wrist joint injury and damage, its surgical plan is determined, and its joint prosthesis is selected.

Imported joint prostheses currently in clinical use are mostly designed with non-limiting or limiting types, which often cannot meet the anatomical characteristics, functional activities and mechanical relationships of the wrist joint, and have doubtable stability.

Due to the above reasons, after years of investigation and clinical research, the present inventor has designed a combined semi-limiting multipolar artificial wrist joint, which can overcome the above shortcoming(s).

### CONTENTS OF THE INVENTION

In order to overcome the above-mentioned problems, the inventor of the present invention has conducted intensive research and designed a combined semi-limiting multipolar artificial wrist joint. It can also be called “semi-limiting bipolar artificial wrist joint”, and be selected and assembled based on the specific pathological conditions of wrist bone destruction. For instance, the metacarpal bone articular surface is shown to be complete and the bone quality is good, and most of the far carpal bones (including small polyangular bones and head bones) can be preserved, while the proximal carpal bones (scaphoid and lunate) and the articular surface of the carpal radius are broken, and the proximal carpal bones are to be excised. The artificial wrist joint can also be assembled in another way, which is called “semi-limiting multipolar artificial wrist joint”, wherein the pathologically damaged double carpal bones are excised to form an inter-matching assembly of palm, wrist and radius joints, and flexibility, convenience and ideal surgical results can be brought to the surgeon.

Specifically, the present invention is to provide a combined semi-limiting bipolar artificial wrist joint, which includes two carpal implant needles 1.

The carpal implant needle 1 is an implant of distal-row retained carpus.

The carpal implant needle 1 is mounted on a shaft 2.

A shaft sleeve 3 is sleeved on the outside of the shaft 2.

The shaft 2 and the shaft sleeve 3 can perform a joint axial movement.

The present invention also provides a combined semi-limiting multipolar artificial wrist joint, which includes a spherical cylinder 6 having a tilted neck at the proximal end of metacarpal bones.

A second platform 61, a second ball neck 62, and a second ball head 63 are provided on the spherical cylinder 6.

Wherein, the second ball neck 62 of the spherical cylinder 6 and the axis of the spherical cylinder 6 form an obliquity of 8-15 degrees.

The beneficial effects of the present invention include:

(1) The combined semi-limiting multipolar artificial wrist joint is composed of the joint ball, the socket lining, and the joint socket. The joint ball and the socket lining are assembled by a super-radius process of the spherical diameter and the socket inner diameter. The socket is in a state of inward contraction around the lining, and the sphere is not easy to fall out, and the whole is assembled with its axial amplitude to leave a certain distance of contraction space, so that the overall joint in work state has the buffer effect of axial stretching, contraction and micro-motion adjustment. The joint cooperates with the soft tissues contracted in a multi-directional and stable simulation of physiological work.

(2) The combined semi-limiting multipolar artificial wrist joint conforms to the physiological and anatomical relationship of the wrist joint, and is more similar to the complex sports medicine characteristics of the wrist joint.

(3) The combined semi-limiting multipolar artificial wrist joint is easy to be assembled, and can move flexibly under certain stress conditions, and achieve multi-directional exercise effects.

(4) The combined semi-limiting multipolar artificial wrist joint is highly versatile, and its combined function can be randomly selected according to the pathological condition of the wrist bone tissue, and the hemicarpal bones and bilateral carpal bones, which will bring a better success rate to the operation.

(5) The surgery of the combined semi-limiting multipolar artificial wrist joint is easy to operate, easy to assemble, and very convenient for promotion and application.

### DESCRIPTION OF FIGURES

FIG. 1 shows a schematic diagram of the overall assembly of a combined semi-limiting bipolar artificial wrist joint according to a preferred embodiment of the present invention;

FIG. 2 shows an exploded schematic diagram of the assembly sequence of a combined semi-limiting bipolar artificial wrist joint according to a preferred embodiment of the present invention in the front and side planes;

FIG. 3 shows a schematic diagram of the overall assembly of a combined semi-limiting multipolar artificial wrist joint according to a preferred embodiment of the present invention;

FIG. 4 shows an exploded schematic diagram of the assembly sequence of a combined semi-limiting multipolar artificial wrist joint according to a preferred embodiment of the present invention;

FIGS. 5-9 respectively show schematic structural diagrams of various components in a combined semi-limiting bipolar artificial wrist joint according to a preferred embodiment of the present invention;

FIGS. 10-14 respectively show schematic structural diagrams of various components in a combined semi-limiting multipolar artificial wrist joint according to a preferred embodiment of the present invention.

### DESCRIPTION OF THE REFERENCE SIGNS

1—a carpal implant needle
2—a shaft
3—a shaft sleeve
4—an articulation movable connector
41—a shaft frame
42—a joint socket
43—a first socket lining
44—a first bulge
5—a spherical cylinder
51—a first platform
52—a first ball neck
53—a first ball head
6—a cylinder having a tilted neck and a ball at the proximal end of metacarpal bones
61-a second platform
62-a second ball neck
63-a second ball head
7—a second socket lining
71—a second bulge
8—a dual articulation movable connector

### SPECIFIC EMBODIMENTS FOR CARRYING OUT THE INVENTION

The present invention will be further described in detail below through the drawings and embodiments. Through these descriptions, the characteristics and advantages of the present invention will become clearer.

The term “exemplary” herein means “serving as an example, embodiment, or illustration.” Any embodiment described herein as “exemplary” need not be construed as being superior to or better than other embodiments. Although various aspects of the embodiments are shown in the drawings, unless otherwise noted, the drawings are not necessarily drawn to scale.

Severe wrist injuries, comminuted fractures of the wrist, malunion, rheumatoid arthritis involving destruction of the wrist joints, unexplained carpal necrosis and wrist joint tumors have become common diseases in specialist clinics and are likely to cause patients deformity, pain and dysfunction of the joints and seriously affect their quality of life and bring great pain. For this reason, we have designed a combined semi-limiting multipolar artificial wrist joint which is divided into two assembly methods through many years of clinical observation and careful research. The main body of these two assembly methods can be matched and exchanged with each other and can be flexibly selected and applied according to the disease condition.

According to the present invention, the combined semi-limiting multipolar artificial wrist joint can be divided into two assembly methods to facilitate the operation success of the surgeon.

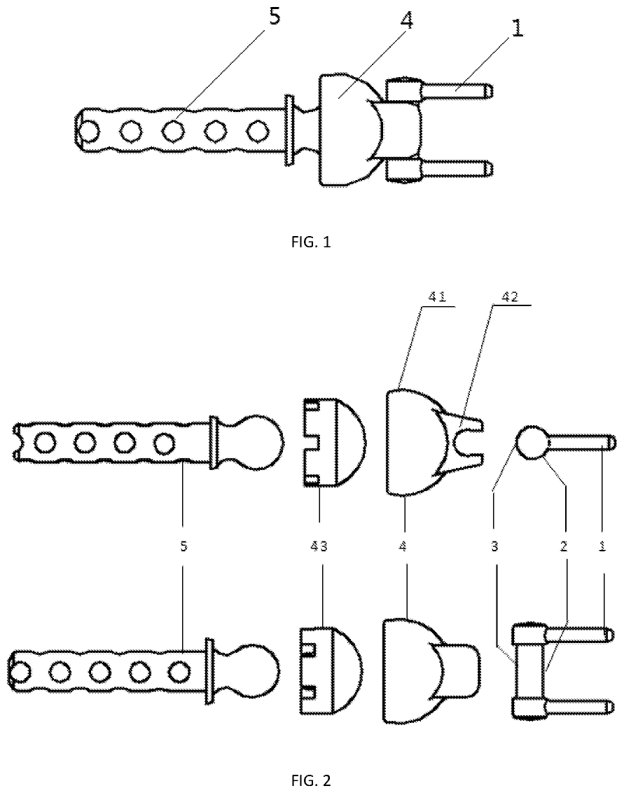

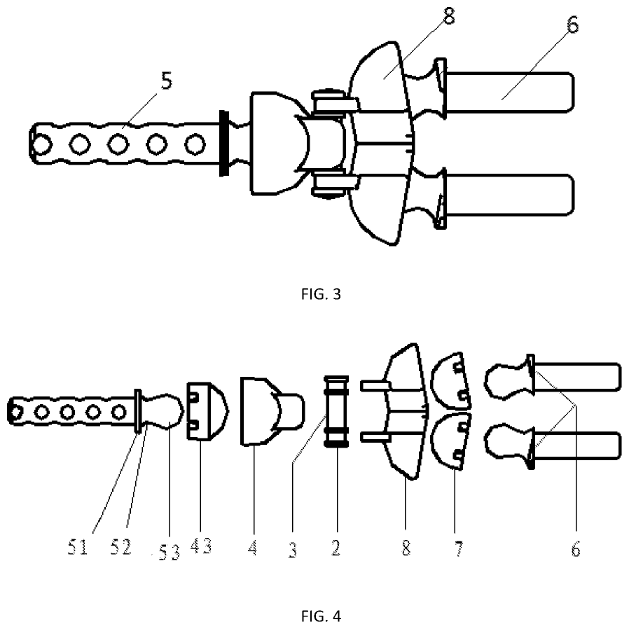

Preferably, in an assembling method, only the proximal carpal bones of the wrist joints need to be excised. As shown in FIGS. 1 and 2, two carpal implant needles shown by reference number 1 are designed as an implant of the distal carpal bone retained. The proximal end is mechanically connected to the two ends of the joint shaft, and can be fixed by a buckle or by an interference fit.

Wherein reference numerals 2 and 3 are shaft and shaft sleeve that can perform joint axial movement. Shaft 2 is made of metal material, and the shaft sleeve 3 that fits on the surface of shaft 2 is made of non-metal material, so that the wear rate between the shaft 2 and the shaft sleeve 3 can be reduced, and the service life of the wrist joint is improved.

The sleeve 3 is sleeved on the middle of the shaft 2, and the carpal implant needles 1 are installed at both ends of the shaft 2. There is a certain distance between the sleeve and the carpal implant needle 1 to avoid interference friction with each other.

The reference number 4 shows an articulation movable connecter. One end of the articulation movable connecter is provided with a joint shaft frame 42, which is assembled with the above-mentioned shaft and sleeve. That is, the shaft 2 and its sleeve 3 are half embedded and inserted into the joint shaft frame 42. The joint shaft frame 42 includes a concave fixed jaw, and the sleeve 3 and the shaft 2 are embedded in the fixed jaw, so that the sleeve 3 and the shaft 2 are installed and fixed to the articulating movable connecter 4.

The other end of the articulation movable connecter is a hemispherical concave articular socket 41 in which a socket lining 43 is installed, and the socket lining 43 is made of non-metallic material.

Reference numeral 5 is a spherical cylinder, which is a distal implant of the radius. The cylinder itself can be a solid cylinder or a hollow cylinder. The surface of the hollow cylinder may be sprayed with biological material. The distal end of the spherical cylinder 5 is provided with a first platform 51 with a 90° angle to the axis of the spherical cylinder 5, that is, the first platform 51 is perpendicular to the spherical cylinder. A first ball neck 52 and a first ball head 53 are arranged on the upper end of the first platform 51, and the first ball head 53 is combined with the joint socket 41 by an over-radius press-fit, that is, the depth value of the ball head 53 inserted into the articular socket lining 43 is greater than the radius value of the ball head 53. After the first ball head in the shape of a spherical shape is installed in the first socket lining, it can rotate relative to the first socket lining to realize the joint-like function. The ball neck 52 is cylindrical and used to connect the first ball head 53 and the spherical cylinder 5.

The overall shape of the first socket lining 43 is hemispherical, and the hemispherical section is recessed inward to provide a groove for accommodating the first ball head 53 on the spherical cylinder 5, and the open end of the groove is provided with a resilient bulge 44 which faces the center of the sphere. When the first ball head is inserted into the groove, the bulge 44 is squeezed to make the bulge 44 elastically deform, thereby the ball head can enter the groove successfully. After the ball head has completely entered the groove, the bulge 44 returns to its original shape and becomes close to the first ball neck, preventing the ball head 51 from coming out of the groove.

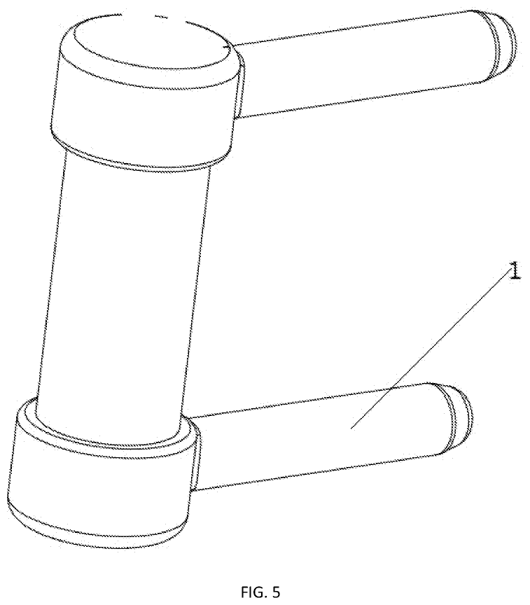

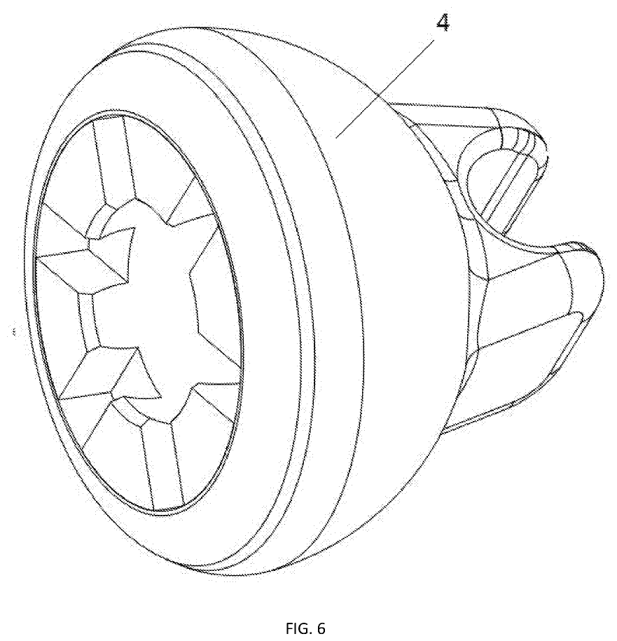

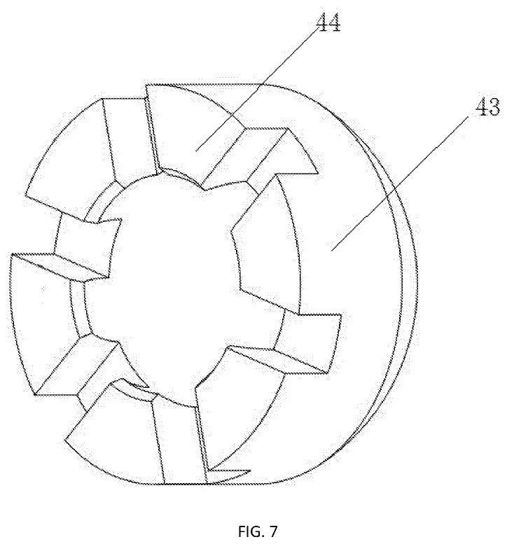

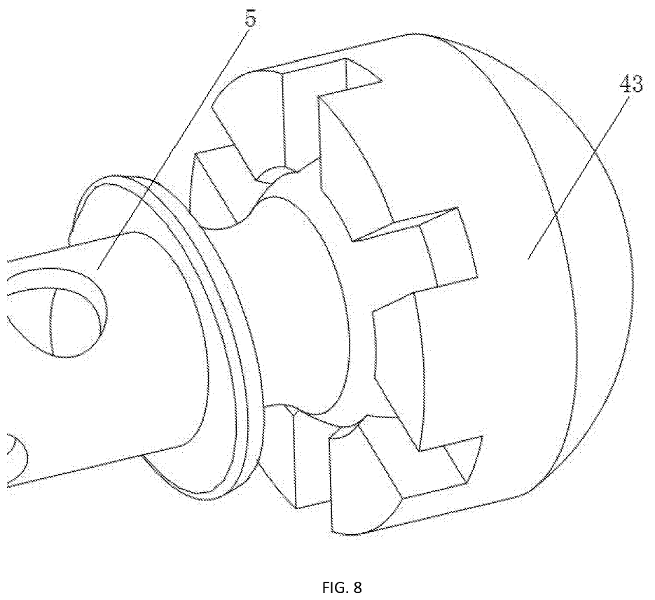

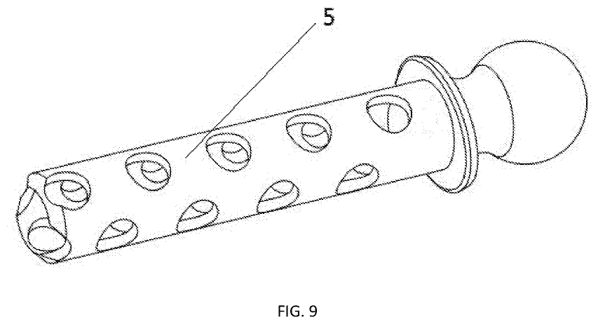

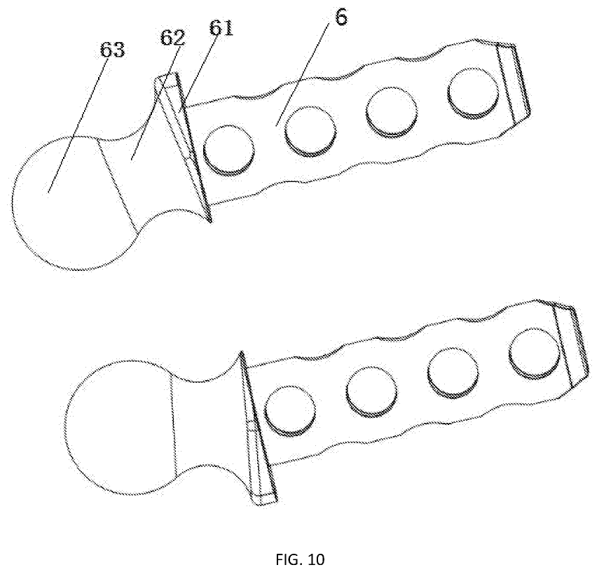

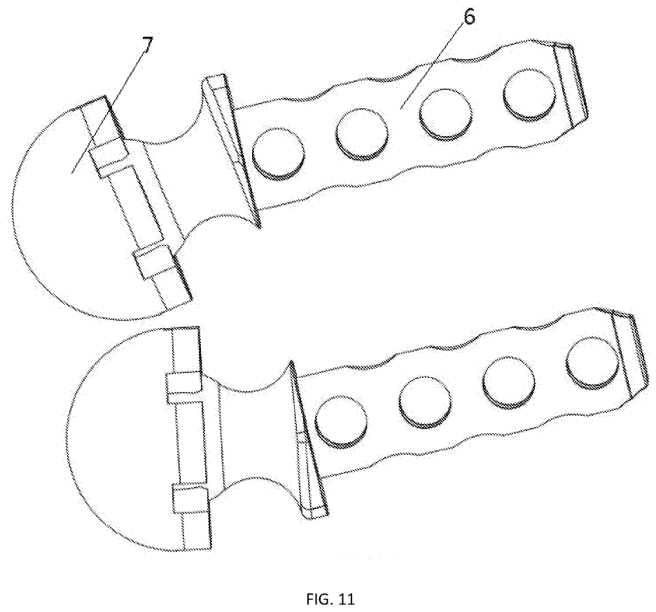

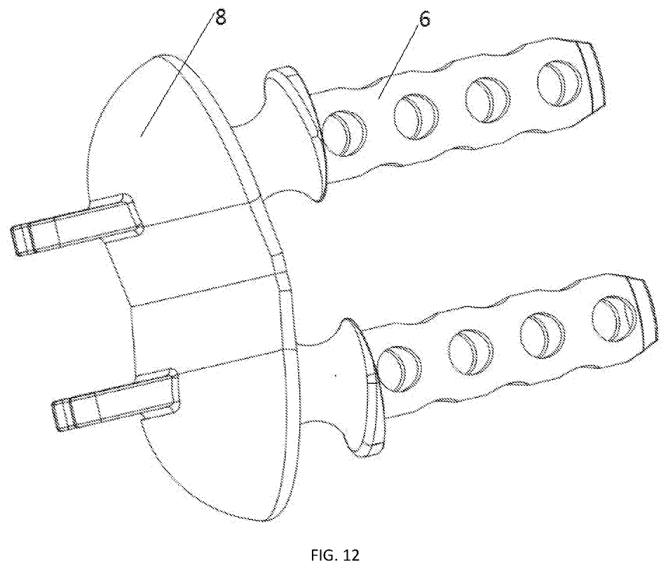

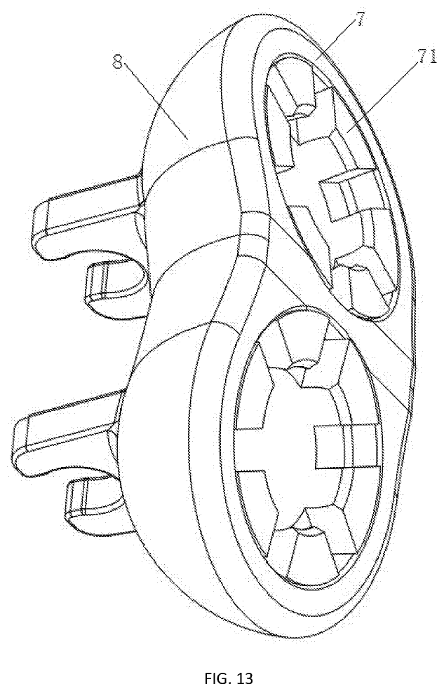

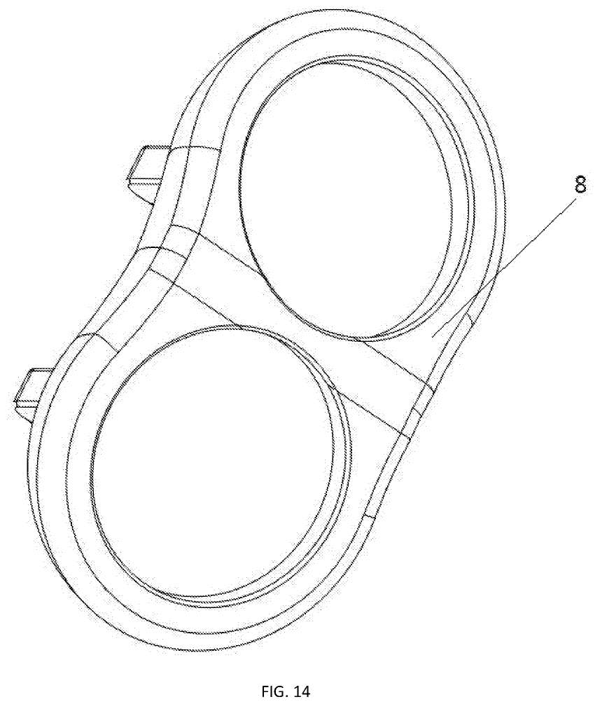

Preferably, in another assembly method, it is also a semi-limiting multipolar wrist joint, which is suitable for the surgical correction of double-row partial carpal resection. Two spherical cylinders corresponding to metacarpal bones, a dual articulation movable connector, and the corresponding inner lining are press-fitted and assembled to the proximal carpal and radius end connector in the same manner as the aforementioned bipolar wrist joint prosthesis to form a palm wrist joint, an intercarpal joint and a carpal radius joint which constitute a multipolar wrist joint with multi-directional freedom, as shown in FIG. 3 and FIG. 4. The above characteristics can be maintained while the need for the orthopedic plan of double-row partial carpal resection is satisfactorily met.

According to the pathological changes of wrist bone injury and breakage, it is necessary to remove double row carpal bone for the multipolar carpal joint prosthesis. As shown in FIGS. 3 and 4, it includes a spherical cylinder 6 having a tilted neck at the proximal end of metacarpal bones. Its shape and process are the same as the previously described for the carpal implant needle. The difference lies in a second ball neck 62 of the spherical cylinder 6 and the axis of the spherical cylinder 6 form an obliquity of 8-15 degrees, preferably 10 degrees. The inventor found that when the angle is set to an obliquity of 10 degrees, it is most suitable for the anatomical morphology of the joint at this position, and can also facilitate matching and association with human femoral tissue and other components on the prosthesis.

A second platform 61, a second ball neck 62, and a second ball head 63 are provided on the spherical cylinder 6. The surface of the second platform 61 facing the spherical cylinder 6 forms an angle of 90 degrees with the axis of the spherical cylinder 6, and the other surface is perpendicular to the axis of the second ball neck 62.

The reference number 7 still refers to a socket lining, which is called a second socket lining 7 here.

The second ball head has a spherical shape and can rotate relative to the second socket lining after being installed in the second socket lining to realize the joint-like function. The second ball neck 62 is cylindrical and is used to connect the second ball head 63 and the spherical cylinder 6.

The second socket lining 7 is hemispherical as a whole, and the hemispherical section is recessed inward to provide a groove for accommodating the second ball head 63 on the spherical cylinder 6, and the open end of the groove is provided with a resilient second bulge 71 which faces the center of the sphere. When the second ball head is inserted into the groove, the second bulge 71 is squeezed to make it elastically deform, thereby the second ball head can enter the groove successfully. After the second ball head has completely entered the groove, the second bulge 71 returns to its original shape and becomes close to the second ball neck 62, preventing the second ball head 61 from coming out of the groove.

Reference numeral 8 denotes a dual articulation movable connector, and its moving end is designed as integration of two sockets which are angled in the opposite directions and press fit with the spherical cylinder 6 angled. A shaft and a sleeve as previously described are provided on the other end of the dual articulation movable connector. Preferably, the two sockets angled in the opposite directions means that the opening surfaces of the two sockets are not parallel, and form an angle is 150 to 170 degrees, preferably 170 degrees, so that both the spherical cylinder 6 and the spherical cylinder 5 installed on the dual articulation movable connector 8 are parallel. That is to say, the dual articulation movable connector 8 can replace the articulation movable connector 4 to perform wrist joint replacement when needed.

This assembly method still includes the shaft 2 and the sleeve 3 described above, and is also provided with a spherical cylinder 6 similar to the spherical cylinder 5. Their functions and assembly method are basically the same as those in the previous assembly method. They can be selected and assembled conveniently according to the pathological conditions and the surgical needs, and satisfactory surgical results can be achieved.

According to the specific pathological conditions of the carpal bone during the operation, the above-mentioned two prosthesis assembly methods can be selected randomly, are mutually compatible, and have strong flexibility. They effectively provide the reliability of the operation, and the assembly of various components is simple and easy. This semi-limiting multipolar artificial wrist joint further highly simulates the complex physiological functions of the human wrist joint.

The above describes the present invention in combination with preferred embodiments, but these embodiments are only exemplary and merely serve for illustration. On this basis, various replacements and improvements can be made to the present invention, all of which fall within the protection scope of the present invention.

What is claimed is:

1. A combined semi-limiting bipolar artificial wrist joint, characterized in that the artificial wrist joint includes two carpal implant needles (1);
the carpal implant needle (1) is an implant of distal-row retained carpus;
the carpal implant needle (1) is mounted on a shaft (2);
a shaft sleeve (3) is sleeved on the outside of the shaft (2);
the shaft (2) and the shaft sleeve (3) can perform a joint axial movement.

2. The combined semi-limiting bipolar artificial wrist joint according to claim 1, wherein:
the shaft (2) is made of metal material, and the shaft sleeve (3) is made of non-metal material.

3. The combined semi-limiting bipolar artificial wrist joint according to claim 1, characterized in that:
the shaft (2) and the shaft sleeve (3) are both installed on the articulation movable connector (4).

4. The combined semi-limiting bipolar artificial wrist joint according to claim 3, characterized in that,
a joint shaft frame (42) matched with the shaft (2) and the shaft sleeve (3) is provided on an end surface of one side of the articulation movable connector (4);
on the other side of the articulation movable connector (4) is a hemispherical concave joint socket (41) in which a first socket lining (43) is installed; preferably, the first socket lining (43) is made of non-metallic material.

5. The combined semi-limiting bipolar artificial wrist joint according to claim 4, characterized in that,
the artificial wrist joint further includes a spherical cylinder (5) which is a distal implant of the radius, and the spherical cylinder (5) itself can be a solid cylinder or a hollow cylinder, preferably, a hollow cylinder, more preferably, a hollow cylinder whose surface is sprayed with a layer of biological material.

6. The combined semi-limiting bipolar artificial wrist joint according to claim 5, characterized in that,
the distal end of the spherical cylinder (5) is provided with a first platform (51) with a 90° angle to the axis of the spherical cylinder (5), and a first ball neck (52) and a first ball head (53) are arranged on the upper end of the platform, and the first ball head (53) is combined with the joint socket (41) by an over-radius press-fit.

7. A combined semi-limiting multipolar artificial wrist joint, characterized in that the artificial wrist joint includes a spherical cylinder (6) having a tilted neck at the proximal end of metacarpal bones;
a second platform (61), a second ball neck (62), and a second ball head (63) are provided on the spherical cylinder (6);
the second ball neck (62) of the spherical cylinder (6) and the axis of the spherical cylinder (6) form an obliquity of 8-15 degrees.

8. The combined semi-limiting multipolar artificial wrist joint according to claim 7, characterized in that,
the combined semi-limiting multipolar artificial wrist joint further includes a dual articulation movable connector (8) inside which second socket lining (7) is installed, and
the spherical cylinder (6) is matched with the second socket lining (7).

9. The combined semi-limiting multipolar artificial wrist joint according to claim 8, characterized in that,
the moving end of the dual articulation movable connector (8) is an integration of two sockets which are angled in the opposite directions and press fit with the spherical cylinder (6) angled;
and a shaft (2) and a shaft sleeve (3) are provided on the other end of the dual articulation movable connector (8).

10. The combined semi-limiting multipolar artificial wrist joint according to claim 9, characterized in that,
the shaft (2) and the shaft sleeve (3) can perform a joint axial movement;
the shaft (2) and the shaft sleeve (3) are both installed on the articulation moveable connector (4),
a joint shaft frame (42) matched with the shaft rod (2) and the shaft sleeve (3) is provided on one end surface of the articulation moveable connector (4);
the other side of the articulation moveable connector (4) is provided with a joint socket (41) in which a first socket lining (43) is installed.
